(19) United States
(12) Patent Application Publication
(10) Pub. No.: US 2025/0154499 A1
(43) Pub. Date: May 15, 2025

(54) METHOD FOR PREPARING RANDOM SGRNA FULL-COVERAGE GROUP OF TARGET SEQUENCE

(71) Applicant: YEASEN BIOTECHNOLOGY (SHANGHAI) CO., LTD., Shanghai (CN)

(72) Inventors: Dongliang SONG, Shanghai (CN); Qian LIU, Shanghai (CN); Cheng HUANG, Shanghai (CN); Ce HOU, Shanghai (CN); Man WANG, Shanghai (CN); Rui SUN, Shanghai (CN); Jingjing CHEN, Shanghai (CN); Zhen CAO, Shanghai (CN)

(73) Assignee: YEASEN BIOTECHNOLOGY (SHANGHAI) CO., LTD., Shanghai (CN)

(21) Appl. No.: 18/722,836

(22) PCT Filed: Dec. 20, 2022
(86) PCT No.: PCT/CN2022/140314

§ 371 Date: Jun. 21, 2024

(30) Foreign Application Priority Data
Dec. 21, 2021 (CN) 202111573668.1

(51) Int. Cl. C12N 15/10

(52) U.S. Cl. CPC C12N 15/1082

(57) ABSTRACT

Provided is a method for preparing a random sgRNA full-coverage group of a target sequence. The method comprises: cleaving a PAM region of a target sequence using a restriction enzyme; linking same to an sgRNA skeleton; acquiring a sequence targeting a protospacer region by means of a restriction enzyme site having a collateral activity on the sgRNA skeleton; linking same to a T7 promoter; acquiring an sgRNA library with the T7 promoter by means of amplification; and acquiring an sgRNA library of the target sequence by means of in-vitro transcription. Also disclosed are a method for preparing an sgRNA library of ribosomal RNA, a method for removing ribosomal RNA from the RNA library, and a method for removing human whole genomes from a host genome. The method has the advantages of low cost, simple manufacture, uniform coverage, low preference, no limitation on the length of a target sequence, no need for design of sgRNAs in large quantities, etc.

### TECHNICAL FIELD

The present application belongs to the field of biotechnology and, in particular, relates to a method for preparing a random sgRNA full-coverage group of target sequence.

### BACKGROUND

Since its development, CRISPR gene editing technology has been widely applied to various fields such as gene therapy, in vitro diagnosis, gene capture and target gene removal, which won the 2020 Nobel Prize in Physiology or Medicine and is an efficient and practical technology. A practical CRISPR system mainly includes two parts: one is a Cas protein with two endonuclease active sites and responsible for cleaving two DNA strands at a target site, and the other is single guide RNA (sgRNA) having a sequence for pairing with DNA at the target site and a Cas protein binding sequence, which isresponsible for recruiting the Cas protein and guiding the Cas protein to bind to the complementary paired target site. In the CRISPR system, the Cas protein first binds to the sgRNA to form a Cas-sgRNA complex and performs retrieval on the DNA. When a region (protospacer) for complementary pairing with the sgRNA is retrieved and an NGG sequence (PAM sequence) exists at the 3′ end of the protospacer, the Cas protein unwinds the target site so that the unwound double-stranded DNA enters a DNA cleavage active domain of the Cas protein, and the Cas protein cleaves the double-stranded DNA so that the double-stranded DNA breaks. The broken DNA is repaired in a DNA damage repairing manner such as homologous recombination (HR) or non-homologous end joining (NHEJ) to complete the editing of a target gene. Current Cas proteins used for commercial applications mainly include Cas9, Cas12, Cas13, Cas14, and variants thereof. Different Cas proteins identify different PAM sequences and have different requirements for protospacer lengths. Therefore, different CRISPR systems have different application scenarios.

Besides the purification and preparation of the Cas protein, the in vitro construction and synthesis of the sgRNA is also an important part for CRISPR commercial applications. In a conventional method, a target sgRNA primer including a T7 promoter is synthesized in a primer synthesis manner, a full-length sgRNA skeleton template is obtained by an overlap polymerase chain reaction (PCR), and the required sgRNA is obtained through in vitro transcription. This method consumes a short time and has a low cost and high controllability and has been commercialized on a large scale and become the main form of in vitro preparation of the sgRNA. However, the method still has the problem of low throughput. Gene capture or removal generally needs to be performed on a genome with a large region and requires the coverage of 1 Mbp Jong DNA or even the entire complete genome DNA. This requires the design and synthesis of tens of millions of sgRNAs. The design and synthesis of the sgRNA are a very large challenge in both cost and technology.

### SUMMARY

In a first aspect, the present application provides a method for preparing a random sgRNA full-coverage group of a target sequence, which comprises:
(1) cleaving a sample DNA using a restriction enzyme for identifying a PAM sequence and flattening an end, wherein the flattening refers to removing 3′ and 5′ single-stranded overhangs of a double-stranded DNA to generate blunt ends;
(2) ligating an sgRNA skeleton to a flattened 3′ end of the double-stranded DNA obtained in step (1), wherein the sgRNA skeleton comprises a collateral activity restriction enzyme sit;
(3) cleaving a ligation product in step (2) using the collateral activity restriction enzyme, acquiring a protospacer DNA, and phosphorylating a 5′ end of the protospacer DNA;
(4) ligating a sequence of a T7 promoter to the 5′ end of the protospacer DNA;
(5) obtaining a T7 promoter-containing sgRNA library template through amplification; and
(6) obtaining an sgRNA library through in vitro transcription.

Preferably, the restriction enzyme in step (1) is one of SerFI, MspI, HpaII, BstNI, BfaI, or DdeI, or a mixture of multiple selected therefrom.

Preferably, in step (1), the end is flattened using Mung Bean Nuclease.

Preferably, the sgRNA skeleton in step (2) is a double-stranded DNA formed through complementary pairing of two single-stranded DNAs, wherein a forward sequence is/rApp/-CGGTTGGAGCTAGAAATAGCAAGTCAACCTAACGCTAGTCCGTTATCAACTTG AAAAAGTGGCACCGAGTCGGTGCTTTT-/NH2C6/(SEQ ID NO: 5), and a reverse sequence is/NH2C6/-AAAAGCACCGACTCGGTGCCACTTTTTCAAGTTGATAACGGACTAGCGTTA GGTTGACTTGCTATTTCTAGCTCCAACC-/ddG/(SEQ NO: 6), with an MmeI restriction site; or a is forward sequence/TApp/-CTGCTGGAGCTAGAAATAGCAAGTCAGCATAACGCTAGTCCGTTATCAACTTG AAAAAGTGGCACCGAGTCGGTGCTTTT-/NH2C6/(SEQ NO: 7), and a reverse sequence is/NH2C6/-AAAAGCACCGACTCGGTGCCACTTTTTCAAGTTGATAACGGACTAGCGTTA TGCTGACTTGCTATTTCTAGCTCCAGCA-/ddG/(SEQ NO: 8), with an EcoP15I restriction site.

Preferably, in step (2), the sgRNA skeleton is ligated to the double-stranded DNA using a T4 DNA ligase mutant K159L.

Preferably, the collateral activity restriction enzyme in step (3) is MmeI; and the T7 promoter in step (4) has a forward sequence of/NH2C6/-TTCTAATACGACTCACTATAGGNN (SEQ NO: 9) and a reverse sequence of/ddC/-CTATAGTGAGTCGTATTAGAA-/NH2C6/(SEQ NO: 10).

Preferably, the collateral activity restriction enzyme in step (3) is EcoP15I; and the T7 promoter in step (4) has a forward sequence of/NH2C6/-TTCTAATACGACTCACTATAGG (SEQ NO: 11) and a reverse sequence of/ddN/-NCTATAGTGAGTCGTATTAGAA-/NH2C6/(SEQ NO: 12).

Preferably, in step (4), the T7 promoter is ligated using a T4 DNA ligase.

Preferably, in step (5), library amplification is performed using a primer with a forward sequence of TTCTAATACGACTCACTATAGG (SEQ NO: 13) and a reverse sequence of AAAAGCACCGACTCGGTGCC (SEQ NO: 14).

Preferably, in step (6), transcription is performed using a T7 RNA polymerase, and the sgRNA library is recovered using RNA recovery magnetic beads.

In a second aspect, the present application provides a method for preparing an sgRNA library of ribosomal RNA, which comprises:
A) preparing 18S and 28S full-length cDNA through reverse transcription;
B) obtaining 18S and 28S full-length double-stranded DNA through PCR amplification; and
C) using the 18S and 28S full-length double-stranded DNA as a sample DNA, and preparing an sgRNA library that targets and covers 18S and 28S by the preceding method.

In a third aspect, the present application provides a method for removing ribosomal RNA from an RNA library, which comprises:
A) pre-assembling the above-prepared sgRNA library with a Cas9 protein;
B) cleaving the RNA library using the pre-assembled Cas9-sgRNA; and
C) amplifying and sequencing the RNA library.

In a fourth aspect, the present application provides a method for removing a human whole genome from a host genome, which comprises:
A) using human whole genome DNA as a sample DNA, and preparing an sgRNA library that targets and covers the human whole genome DNA by the preceding method;
B) pre-assembling the prepared sgRNA library with a Cas9 protein;
C) cleaving a DNA library of the host genome using the pre-assembled Cas9-sgRNA; and
D) amplifying and sequencing the DNA library.

The present application has the beneficial effects described below.

The present application provides random sgRNA preparation of the target sequence (RPTS) so that a group of all sgRNAs that randomly cover a target region can be prepared at a time. The RPTS has the following principle and process: the PAM region of the target sequence is cleaved using the restriction enzyme: the sgRNA skeleton is ligated; a sequence that targets a protospacer is acquired through the collateral activity restriction enzyme site on the sgRNA skeleton: the T7 promoter is ligated: the sgRNA library with the T7 promoter is obtained through amplification; and the sgRNA library of the target sequence is obtained through in vitro transcription. The RPTS has the advantages of a low cost, simple production, uniform coverage, a low preference, independence of the length of the target sequence and no need to design sgRNA on a large scale. The present application further provides processes of applications of the RPTS to the removal of ribosomal RNA and the removal a human genome from the host genome so that limitations in the applications of the CRISPR technology to these fields are overcome.

### BRIEF DESCRIPTION OF DRAWINGS

FIG. 1 is a diagram of binding of normal sgRNA to a target site.

FIG. 2 shows a modified sgRNA skeleton comprising an MmeI restriction site.

FIG. 3 shows a modified sgRNA skeleton comprising an EcoP15I restriction site.

FIG. 4 is a schematic diagram of the principle and process of RPTS.

FIG. 5 shows a summary of restriction enzyme recognition sites used in RPTS, wherein a shadow indicates a PAM sequence type comprised in a restriction enzyme recognition site.

FIG. 6 shows the distribution of restriction enzyme recognition sites used in RPTS comprised in DNA of 18S rRNA.

FIG. 7 shows amplification results of 18S and 28S cDNA, wherein the left strip represents Marker, the middle strip represents 28S, and the right strip represents 18S.

FIG. 8 shows RPTS library amplification results of 18S/28S DNA and human whole genome DNA (right), wherein the left strip represents Marker, the middle strip represents the 18S/28S DNA, and the right strip represents the human whole genome DNA.

FIG. 9 shows in vitro transcription results of 18S/28S DNA (left) and human whole genome DNA (right), wherein the left strip represents Marker, the middle strip represents the 18S/28S DNA, and the right strip represents the human whole genome DNA.

FIG. 10 shows the library distribution of an 18S/28S RPTS library in an application of CRISPR to rRNA removal. The light line represents the library without RPTS library, and the dark line represents the library with RPTS library.

FIG. 11 shows a removal result of an 18S/28S RPTS library in an application of CRISPR to TRNA removal, which is verified by RNA-seq.

FIG. 12 shows the library distribution of a human whole genome RPTS library in an application of CRISPR to removal a human genome from a host genome. The light line represents library without RPTS library, and the dark line represents the library with RPTS library.

FIG. 13 shows a removal result of a human whole genome RPTS library in an application of CRISPR to removal a human genome from a host genome, which is verified by DNA-seq.

### DETAILED DESCRIPTION

To further describe the specific content of the present application, the present application is described in detail below in conjunction with examples. Operation methods and reagents involved in the examples are well known to those skilled in the art. It is to be understood that the examples described below are intended to describe the present application in detail and not to limit embodiments of the present application. Linker sequences used in the examples and modifications thereof are shown in Table 1, wherein N represents any base of A, T, C, and G.

TABLE 1
Linker sequences and modifications

| | Sequence | | |
|---|---|---|---|
| No. | Name | 5′-3′ | Modification |
| SEQ NO: 1 | 18S-R | TAATGATCCTTCCGCAGGTTCACCTACG | |
| SEQ NO: 2 | 18S-F | TACCTGGTTGATCCTGCCAGTAG | |
| SEQ NO: 3 | 28S-R | GACAAACCCTTGTGTCGAGGGCTGACT | |
| | | T | |
| SEQ NO: 4 | 28S-F | CGCGACCTCAGATCAGACGTG | |
| SEQ NO: 5 | sgM-F | /rApp/-CGGTTGGAGCTAGAAATAGCAAG | The 5′ end is |
| | | TCAACCTAACGCTAGTCCGTTATCAACT | pre-adenylated |
| | | TGAAAAAGTGGCACCGAGTCGGTGCTT | for an rApp |
| | | TT-/NH2C6/ | modification, |
| | | | and the 3′ end |
| | | | is capped with |
| | | | an amino |
| | | | modification |
| | | | NH2C6. |
| SEQ NO: 6 | sgM-R | /NH2C6/-AAAAGCACCGACTCGGTGCCA | The 5′ end is |
| | | CTTTTTCAAGTTGATAACGGACTAGCGT | capped with an |
| | | TAGGTTGACTTGCTATTTCTAGCTCCAA | amino |
| | | CC-/ddG/ | modification |
| | | | NH2C6, and |
| | | | the 3′ end is |
| | | | capped with a |
| | | | dideoxynucleotide |
| | | | modification |
| | | | ddG. |
| SEQ NO: 7 | sgB-F | /rApp/-CTGCTGGAGCTAGAAATAGCAAG | The 5′ end is |
| | | TCAGCATAACGCTAGTCCGTTATCAACT | pre-adenylated |
| | | TGAAAAAGTGGCACCGAGTCGGTGCTT | for an rApp |
| | | TT-/NH2C6/ | modification, |
| | | | and the 3′ end |
| | | | is capped with |
| | | | an amino |
| | | | modification |
| | | | NH2C6. |
| SEQ NO: 8 | sgE-R | /NH2C6/-AAAAGCACCGACTCGGTGCCA | The 5′ end is |
| | | CTTTTTCAAGTTGATAACGGACTAGCGT | capped with an |
| | | TATGCTGACTTGCTATTTCTAGCTCCAGC | amino |
| | | A-/ddG/ | modification |
| | | | NH2C6, and |
| | | | the 3′ end is |
| | | | capped with a |
| | | | dideoxynucleotide |
| | | | modification |
| | | | ddG. |
| SEQ NO: 9 | T7M-F | /NH2C6/-TTCTAATACGACTCACTATAGG | The 5′ end is |
| | | NN | capped with an |
| | | | amino |
| | | | modification |
| | | | NH2C6. |
| SEQ NO: 10 | T7M-R | /ddC/-CTATAGTGAGTCGTATTAGAA-/ | The 5′ end is |
| | | NH2C6/ | capped with a |
| | | | dideoxynucleotide |
| | | | modification |
| | | | ddC, and the 3′ |
| | | | end is capped |
| | | | with an amino |
| | | | modification |
| | | | NH2C6. |
| SEQ NO: 11 | T7E-F | /NH2C6/-TTCTAATACGACTCACTATAGG | The 5′ end is |
| | | | capped with an |
| | | | amino |
| | | | modification |
| | | | NH2C6. |
| SEQ NO: 12 | T7E-R | /ddN/-NCTATAGTGAGTCGTATTAGAA-/ | The 5′ end is |
| | | NH2C6/ | capped with a |
| | | | dideoxynucleotide |
| | | | modification |
| | | | ddN, and the 3′ |
| | | | end is capped |
| | | | with an amino |
| | | | modification |
| | | | NH2C6. |
| SEQ NO: 13 | sgPCR-F | TTCTAATACGACTCACTATAGG | |
| SEQ NO: 14 | sgPCR-R | AAAAGCACCGACTCGGTGCC | |

### Example 1: Design of an sgRNA Skeleton and Process of RPTS

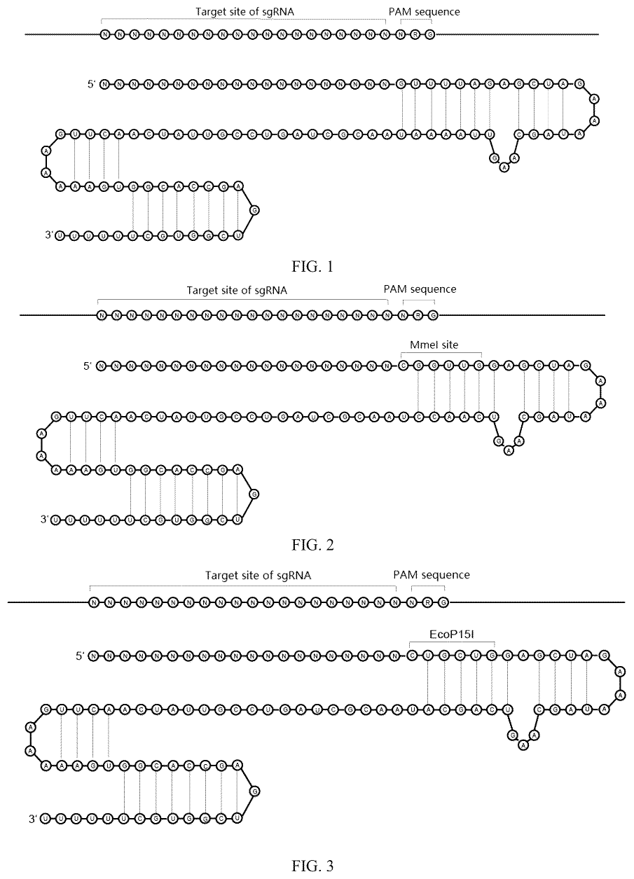

In this example, a conventional sgRNA skeleton was modified with an MmeI or EcoP15I restriction site fused and without changing the structure of sgRNA and the binding to Cas9. The sequences and structures of the modified sgRNA are shown in FIGS. 1 to 3.

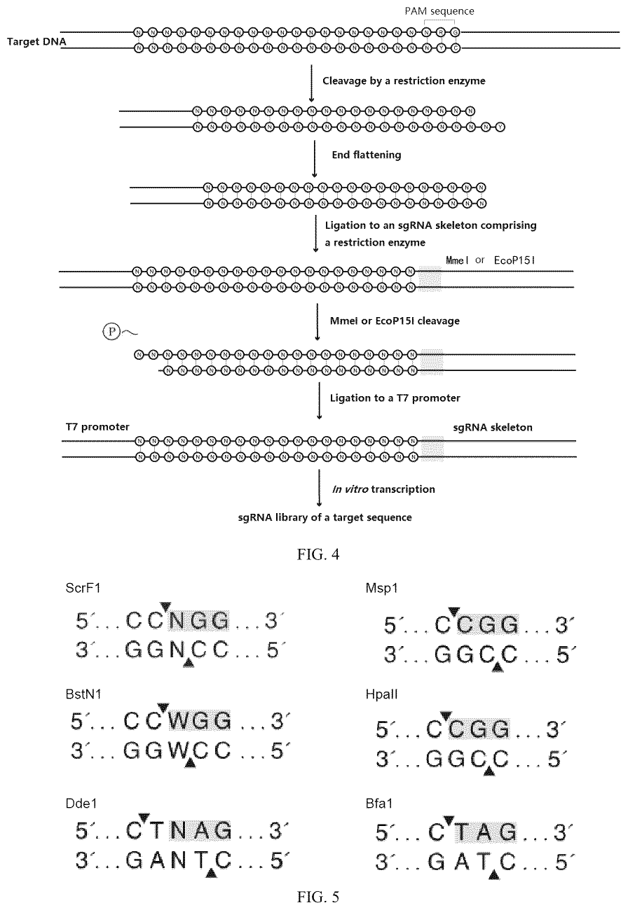

The process of RPTS was tested. The process is shown in FIG. 4.

### (1) Cleavage by a Restriction Enzyme for Identifying a PAM Sequence

TABLE 2
Restriction enzyme digestion system

| Component | Amount |
|---|---|
| PUC19 plasmid DNA | 1 μg |
| 10× rCutSmart buffer | 8 μL |
| ScrFI (NEB) | 5 U |
| MspI (NEB) | 5 U |
| HpaII (NEB) | 5 U |
| BstNI (NEB) | 5 U |
| BfaI (NEB) | 5 U |
| DdeI (NEB) | 5 U |
| Addition of water to | 80 μL |

The reaction was performed overnight at 37° C. After the reaction was completed, 160 μL of Ampure DNA beads (Beckman) was added to recover fragmented DNA. Elution was performed with 45 μL of water.

TABLE 3
End flattening system

| Component | Amount |
|---|---|
| DNA recovered above | 43 μL |
| 10× Mung Bean Nuclease Reaction Buffer | 5 μL |
| Mung Bean Nuclease (NEB) | 20 U |
| Addition of water to | 50 μL |

The reaction was performed for 2 h at 30° C. After the reaction was completed, 100 μL of Ampure DNA beads (Beckman) was added to recover fragmented DNA. Elution was performed with 42 μL of water.

(2) Ligation to the sgRNA Skeleton Comprising a Collateral Activity Restriction Enzyme Site.

Skeleton annealing: sgM-F, sgM-R, sgE-F, and sgE-R were dissolved to 100 μM with a 100 mM NaCl solution, 10 μL of sgM-F and 10 μL of sgM-R were added to a PCR tube, and 10 μL of sgE-F and 10 L of sgE-R were added to another PCR tube. In the same reaction system, one of an MmeI-sgRNA skeleton and an EcoP15I-sgRNA skeleton was selected. The reaction was performed for 5 min at 95° C., with 1° C. decreased per minute. After the reaction was completed, the annealed skeleton was diluted to 10 μM with water.

### Skeleton Ligation:

TABLE 4
Skeleton ligation system

| Component | Amount |
|---|---|
| DNA recovered above | 40 μL |
| 10 μM sgRNA skeleton linker | 5 μL |
| 10× T4 Ligase Reaction Buffer | 5 μL |
| T4 DNA Ligase (K159L) | 2000 U |
| Addition of water to | 50 μL |

The reaction was performed for 1 h at 20° C. After the reaction was completed, 22.5 μL of Ampure DNA beads was added to recover DNA, and linkers not ligated were removed. Elution was performed with 22 μL of water.

### (3) MmeI or EcoP15I Digestion

TABLE 5
MmeI digestion system

| Component | Amount |
|---|---|
| Above-recovered DNA comprising the | 20 uL |
| MmeI-sgRNA skeleton | |
| 10× rCutSmart buffer | 3 μL |
| MmeI | 5 U |
| Addition of water to | 30 μL |

TABLE 6
EcoP15I digestion system

| Component | Amount |
|---|---|
| Above-recovered DNA comprising the | 20 μL |
| EcoP15I-sgRNA skeleton | |
| 10× rCutSmart buffer | 3 μL |
| 10× ATP | 3 μL |
| EcoP15I | 20 U |
| Addition of water to | 30 μL |

The reaction was performed for 2 h at 37° C. After the reaction was completed, 60 μL of Ampure DNA beads was added to recover DNA. Elution was performed with 42 μL of water.

### (4) Ligation to a T7 Promoter

Linker annealing: T7M-F, T7M-R, T7E-F and T7E-R were dissolved to 100 μM with a 100 mM NaCl solution, 10 μL of T7M-F and 10 μL of T7M-R were added to a PCR tube, 10 μL of T7E-F and 10 μL of T7E-R were added to another PCR tube. An appropriate T7 linker was selected according to a type of the above sgRNA skeleton. The reaction was performed for 5 min at 95° C., with 1° C. decreased per minute. After the reaction was completed, the annealed linker was diluted to 10 UM with water.

### Linker Ligation:

TABLE 7
Linker ligation system

| Component | Amount |
|---|---|
| DNA recovered above | 40 μL |
| Corresponding 10 μM T7 linker | 5 μL |
| 10× T4 Ligase Reaction Buffer | 5 μL |
| T4 DNA Ligase | 2000 U |
| Addition of water to | 50 μL |

The reaction was performed for 1 h at 20° C. After the reaction was completed, 22.5 μL of Ampure DNA beads was added to recover DNA, and linkers not ligated were removed. Elution was performed with 22 μl of water.

(5) sgRNA Library Amplification

TABLE 8
sgRNA library amplification system

| Component | Amount |
|---|---|
| DNA recovered above | 20 μL |
| 10 μM sgPCR-F/R | 5 μL |
| Phusion High-Fidelity PCR Master Mix | 25 μL |
| Addition of water to | 50 μL |

After 3 min of denaturation at 98° C., library cycling amplification was performed through 10s of denaturation at 98° C., 20s of annealing at 60° C., and 10s of extension at 72° C. The amplification product was recovered with 70 μl of Ampure DNA beads and eluted with 22 μl . . . of DEPC water.

(6) In Vitro Transcription of the sgRNA Library

TABLE 9
System for in vitro transcription of the sgRNA library

| Component | Amount |
|---|---|
| DNA recovered above | 1 μg |
| 10× Transcription Buffer | 2 μL |
| CTP/GTP/ATP/UTP (100 mM each) | 2 μL |
| T7 RNA Polymerase Mix (Yeasen) | 2 μL |
| Addition of water to | 20 μL |

The system was reacted for 4 h at 37° C., added with 10 U DNase I (TAKARA), and reacted for 1 h at 37° C. 50 μL of Ampure RNA beads (Beckman) was added to recover RNA. A size of sgRNA was detected through agarose gel electrophoresis.

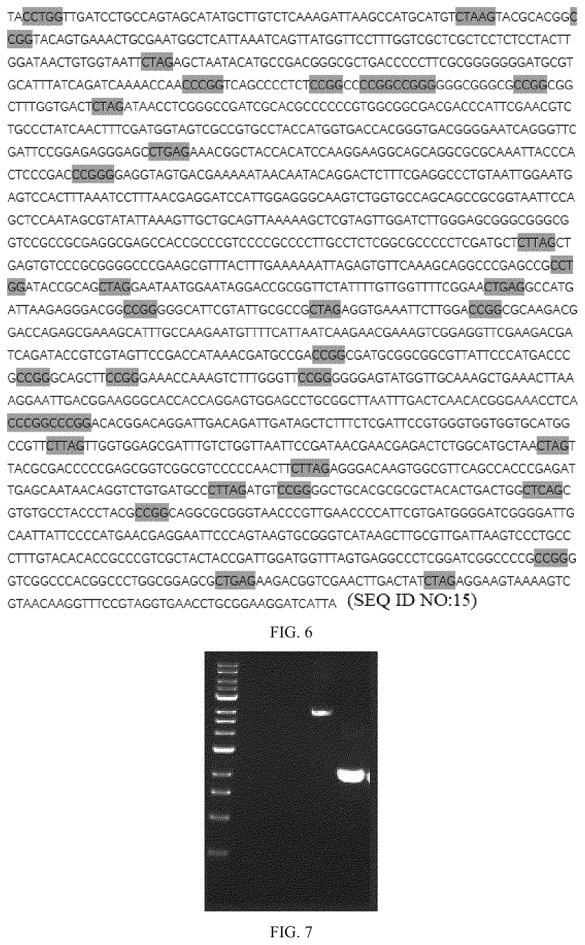

As shown in FIG. 5, a designed combination of restriction enzymes for identifying the PAM sequence (NRG) comprises six restriction enzymes, and six restriction enzyme sites comprise two PAM sequences for the Cas9 protein. Such restriction enzyme sites exist on DNA about every 64 bp, so the prepared sgRNA can randomly cover the whole genome. FIG. 6 shows the distribution of the six restriction enzyme sites (shadows) on 18S rRNA.

### Example 2: Preparation of RPTS of 18S rRNA or 28S rRNA

In this example, a random sgRNA library covering 18S rRNA or 28S rRNA was prepared by RPTS. A specific implementation manner was described below.

### (1) Acquisition of DNA Fragments

TABLE 10
Reverse transcription system

| Component | Amount |
|---|---|
| 293 cell RNA | 1 μg |
| 10 μM reverse transcription primer | 1 μL |
| 18S-R or 28S-R | |
| 10 mM dNTPs | 1 |
| 5 min of reaction at 75° C. | |
| 5× FS Buffer | 4 μL |
| 0.1M DTT | 1 μL |
| SuperScript IV(Thermo) | 2 μL |
| Total volume | 20 μL |

42° C. 15 min, 50° C. 15 min, 55° C. 15 min, 50° C. 15 min, 55° C. 15 min, 70° C. 15 min.

TABLE 11
PCR amplification system

| Component | Amount |
|---|---|
| Above reverse transcription product | 1 μL |
| 10 μM 18S-F/R or 28S-F/R | 5 μL |
| Phusion High-Fidelity PCR Master Mix | 25 μL |
| Addition of water to | 50 μL |

After 3 min of denaturation at 98° C., library cycling amplification was performed through 10s of denaturation at 98° C., 20s of annealing at 60° C., and 3 min of extension at 72° C. The amplification product was recovered with 35 μL of Ampure DNA beads and eluted with 22 μL of DEPC water.

Preparation of the sgRNA library by the RPTS: An 18S sgRNA library or a 28S sgRNA library was prepared according to the method of Example 1.

(3) Preparation of an RNA Library: The RNA Library was Constructed Using a Dual-Mode RNA Library Preparation Kit (12252) of Yeasen Biotechnology. After DNA Linkers were Ligated, the Library was Recovered with 0.6×Ampure DNA Beads.

### (4) Removal of 18S and 28S DNA by CRISPR

TABLE 12
System for preassembly of the sgRNA library with the Cas9 protein

| Component | Amount |
|---|---|
| 18S sgRNA library and 28S sgRNA library | 2-10 μg |
| Cas9 (NEB) | 0.2-0.5 μg |
| 500 mM sodium chloride | 1 μL |
| Total volume | 5 μL |

37° C. 30 min.

TABLE 13
CRISPR cleavage system

| Component | Amount |
|---|---|
| Cas9-sgRNA library | 5 μL |
| RNA library | 1-100 ng |
| 10× NEB buffer 3.1 | 1 μL |
| Total volume | 10 μL |

37° C. 30-90 min, 90° C. 10 min.

### (5) Library Amplification

TABLE 14
Library amplification system

| Component | Amount |
|---|---|
| Above reaction system | 10 μL |
| Index Primer F/R (Yeasen, 12610) | 5 μL |
| 2× Canase PCR mix | 25 μL |
| Addition of water to | 50 μL |

After 3 min of denaturation at 98° C., library cycling amplification was performed through 10s of denaturation at 98° C., 30s of annealing at 60° C., and 30s of extension at 72° C. The amplification product was recovered with 45 μL of Ampure DNA beads and eluted with 22 μL of DEPC water.

After Qsep100 quality detection, the prepared library was sequenced and analyzed on a platform NovaSeq 6000 of Illumina.

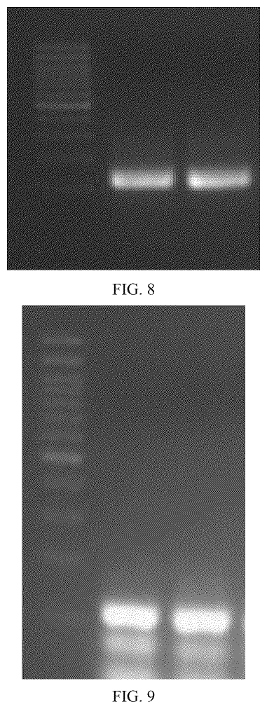

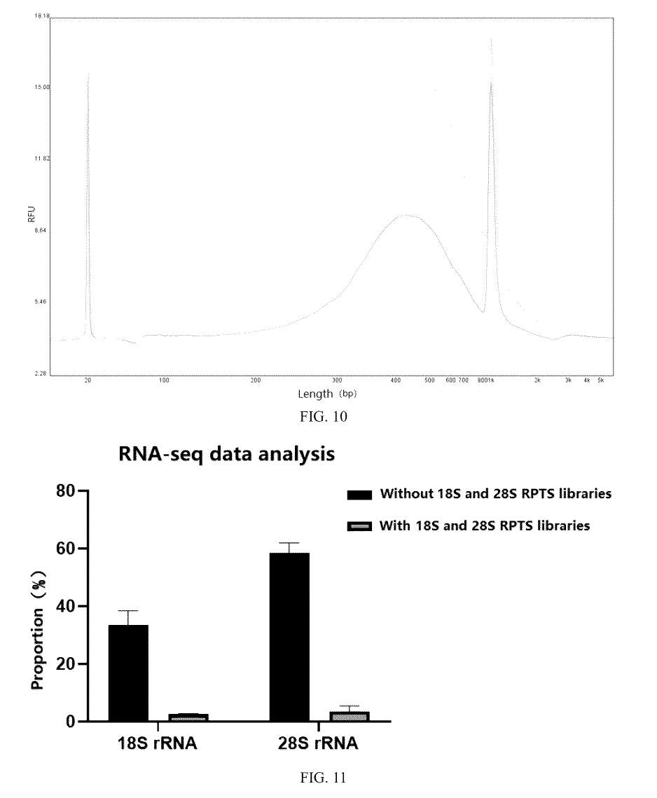

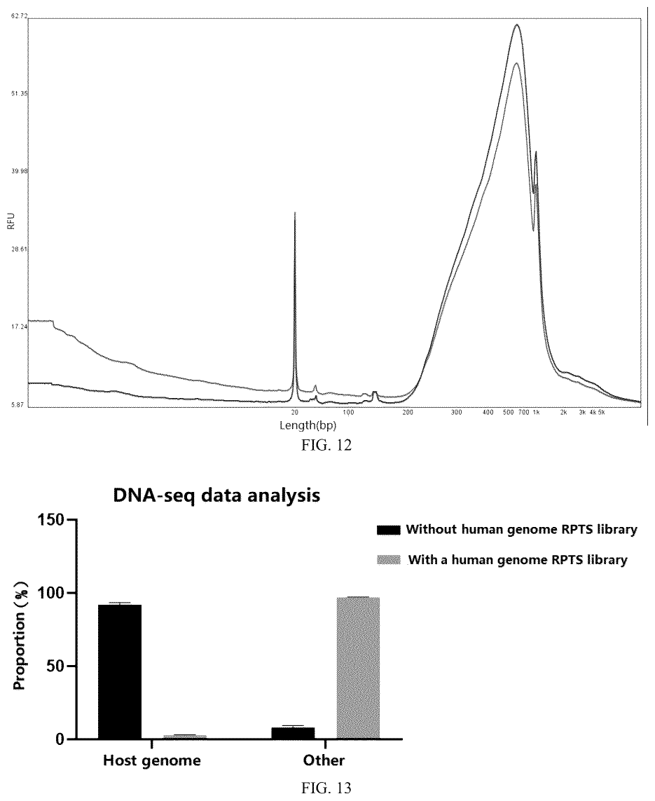

18S or 28S RPTS results are shown in FIGS. 7, 8, and 9. The RPTS technology can successfully construct the 18S or 28S sgRNA library. RNA library construction and sequencing results are shown in FIGS. 10 and 11. The sgRNA library prepared by the RPTS method can effectively remove 18S and 28S rRNA.

### Example 3: Preparation of RPTS of a Human Whole Genome

In this example, a random sgRNA library covering the human whole genome was prepared by RPTS. A specific implementation manner was described below.

(1) Preparation of the sgRNA library by the RPTS: The sgRNA library was prepared according to the method of Example 1, and DNA used was a human genome DNA standard product NA12878 (Coriell).

(2) Preparation of a DNA library: DNA used was a DNA standard product mixture of the human genome DNA standard product NA12878 (Coriell) and an Escherichia coli genome, which were mixed at the ratio of 100:1. The DNA library was constructed using a one-step library preparation kit (12204) of Yeasen Biotechnology. After DNA linkers were ligated, the library was recovered with 0.6×Ampure DNA beads.

(3) Removal of human genome DNA by CRISPR

TABLE 15
System for preassembly of the sgRNA library with the Cas9 protein

| Component | Amount |
|---|---|
| Human genome RPTS library | 2-10 μg |
| Cas9 (NEB) | 0.2-0.5 μg |
| 500 mM sodium chloride | 1 μL |
| Total volume | 5 μL |

37° C. 30 min.

TABLE 16
CRISPR cleavage system

| Component | Amount |
|---|---|
| Cas9-sgRNA library | 5 μL |
| RNA library | 1-100 ng |
| 10× NEB buffer 3.1 | 1 μL |
| Total volume | 10 μL |

37° C. 30-90 min, 90° C. 10 min.

### (5) Library Amplification

TABLE 17
Library amplification system

| Component | Amount |
|---|---|
| Above reaction system | 10 μL |
| Index Primer F/R (Yeasen, 12610) | 5 μL |
| 2× Canace pro PCR mix | 25 μL |
| Addition of water to | 50 μL |

After 3 min of denaturation at 98° C., library cycling amplification was performed through 10s of denaturation at 98° C., 30s of annealing at 60° C., and 30s of extension at 72° C. The amplification product was recovered with 45 μL of Ampure DNA beads and eluted with 22 μL of DEPC water.

After Qsep100 quality detection, the prepared library was sequenced and analyzed on a platform NovaSeq 6000 of Illumina.

DNA library construction and sequencing results are shown in FIGS. 11 and 12. The sgRNA library prepared by the RPTS method can effectively remove a human whole genome from host genome DNA in the DNA library construction process.

To conclude, the present application discloses RPTS (random sgRNA preparation of the target sequence) so that a group of all sgRNAs that randomly cover a target region can be prepared at a time. The RPTS has the following principle and process: the PAM region of the target sequence is cleaved using the restriction enzyme; the sgRNA skeleton is ligated; a sequence that targets a protospacer is acquired through the collateral activity restriction enzyme site on the sgRNA skeleton: the T7 promoter is ligated; the sgRNA library with the T7 promoter is obtained through amplification; and the sgRNA library of the target sequence is obtained through in vitro transcription. The RPTS has the advantages of a low cost, simple production, uniform coverage, a low preference, independence of the length of the target sequence and no need to design sgRNA on a large scale. The present application further discloses processes of applications of the RPTS to the removal of ribosomal RNA and the removal a human genome from the host genome so that limitations in the applications of the CRISPR technology to these fields are overcome.

1. A method for preparing a random sgRNA full-coverage group of target sequence, comprising:
(1) cleaving a sample DNA using a restriction enzyme for identifying a PAM sequence, and flattening end(s);
(2) ligating an sgRNA skeleton to a flattened 3′ end of a double-stranded DNA obtained in step (1), wherein the sgRNA skeleton comprises a collateral activity restriction enzyme site;
(3) cleaving a ligation product in step (2) using the collateral activity restriction enzyme, acquiring a protospacer DNA, and phosphorylating a 5′ end of the protospacer DNA;
(4) ligating a sequence of a T7 promoter to the 5′ end of the protospacer DNA;
(5) obtaining a T7 promoter-containing sgRNA library template through amplification; and
(6) obtaining an sgRNA library through in vitro transcription.

2. The method for preparing a random sgRNA full-coverage group of target sequence according to claim 1, wherein the restriction enzyme in step (1) is one of ScrFI, MspI, HpaII, BstNI, BfaI, or DdeI or a mixture of multiple selected therefrom.

3. The method for preparing a random sgRNA full-coverage group of target sequence according to claim 1, wherein in step (1), the end is flattened using Mung Bean Nuclease.

4. The method for preparing a random sgRNA full-coverage group of target sequence according to claim 1, wherein the sgRNA skeleton in step (2) is a double-stranded DNA formed through complementary pairing of two single-stranded DNAs, wherein a forward sequence is/rApp/-CGGTTGGAGCTAGAAATAGCAAGTCAACCTAACGCTAGTCCGTTATCAACTTG AAAAAGTGGCACCGAGTCGGTGCTTTT-/NH2C6/(SEQ ID NO: 5), and a reverse sequence is/NH2C6/-AAAAGCACCGACTCGGTGCCACTTTTTCAAGTTGATAACGGACTAGCGTTAGG TTGACTTGCTATTTCTAGCTCCAACC-/ddG/(SEQ NO: 6), with an MmeI restriction site: or a forward sequence is/rApp/-CTGCTGGAGCTAGAAATAGCAAGTCAGCATAACGCTAGTCCGTTATCAACTTG AAAAAGTGGCACCGAGTCGGTGCTTTT-/NH2C6/(SEQ NO: 7), and a reverse sequence is/NH2C6/-AAAAGCACCGACTCGGTGCCACTTTTTCAAGTTGATAACGGACTAGCGTTATG CTGACTTGCTATTTCTAGCTCCAGCA-/ddG/(SEQ NO: 8), with an EcoP15I restriction site.

5. The method for preparing a random sgRNA full-coverage group of target sequence according to claim 16, wherein in step (2), the sgRNA skeleton is ligated to the double-stranded DNA using a T4 DNA ligase mutant K159L.

6. The method for preparing a random sgRNA full-coverage group of target sequence according to claim 1, wherein the collateral activity restriction enzyme in step (3) is MmeI; and the T7 promoter in step (4) has a forward sequence of/NH2C6/-TTCTAATACGACTCACTATAGGNN (SEQ NO: 9) and a reverse sequence of/ddC/-CTATAGTGAGTCGTATTAGAA-/NH2C6/(SEQ NO: 10).

7. The method for preparing a random sgRNA full-coverage group of target sequence according to claim 1, wherein the collateral activity restriction enzyme in step (3) is EcoP15I; and the T7 promoter in step (4) has a forward sequence of/NH2C6/-TTCTAATACGACTCACTATAGG (SEQ NO: 11) and a reverse sequence of/ddN/-NCTATAGTGAGTCGTATTAGAA-/NH2C6/(SEQ NO: 12).

8. The method for preparing a random sgRNA full-coverage group of target sequence according to claim 1, wherein in step (4), the T7 promoter is ligated using a T4 DNA ligase.

9. The method for preparing a random sgRNA full-coverage group of target sequence according to claim 1, wherein in step (5), library amplification is performed using a primer pair with a forward sequence of TTCTAATACGACTCACTATAGG and a reverse sequence of AAAAGCACCGACTCGGTGCC.

10. The method for preparing a random sgRNA full-coverage group of target sequence according to claim 1, wherein in step (6), transcription is performed using a T7 RNA polymerase, and the sgRNA library is recovered using RNA recovery magnetic beads.

11. A method for preparing an sgRNA library of ribosomal RNA, comprising:
A) preparing 18S and 28S full-length cDNA through reverse transcription;
B) obtaining 18S and 28S full-length double-stranded DNA through PCR amplification; and
C) using the 18S and 28S full-length double-stranded DNA as a sample DNA, and preparing an sgRNA library that targets and covers 18S and 28S by the method according to claim 1.

12. A method for removing ribosomal RNA from an RNA library, comprising:
A) pre-assembling the sgRNA library prepared according to claim 11 with a Cas9 protein;
B) cleaving the RNA library using the pre-assembled Cas9-sgRNA; and
C) amplifying and sequencing the RNA library.

13. A method for removing a human whole genome from a host genome, comprising:
A) using human whole genome DNA as a sample DNA, and preparing an sgRNA library that targets and covers the human whole genome DNA by the method according to claim 1;
B) pre-assembling the prepared sgRNA library with a Cas9 protein;
C) cleaving a DNA library of the host genome using the pre-assembled Cas9-sgRNA; and
D) amplifying and sequencing the DNA library.


SEQUENCE LISTING (15 sequences)

SEQ ID NO: 1 - DNA, 28 bp, synthetic construct
  source 1..28: mol_type = other DNA
taatgatccttccgcaggttcacctacg

SEQ ID NO: 2 - DNA, 23 bp, synthetic construct
  source 1..23: mol_type = other DNA
tacctggttgatcctgccagtag

SEQ ID NO: 3 - DNA, 28 bp, synthetic construct
  source 1..28: mol_type = other DNA
gacaaacccttgtgtcgagggctgactt

SEQ ID NO: 4 - DNA, 21 bp, synthetic construct
  source 1..21: mol_type = other DNA
cgcgacctcagatcagacgtg

SEQ ID NO: 5 - DNA, 80 bp, synthetic construct
  source 1..80: mol_type = other DNA
  modified_base 1: mod_base = OTHER; note = adenylpyrophosphoryl
  modified_base 80: mod_base = OTHER; note = 5' amino modifier C6
cggttggagctagaaatagcaagtcaacctaacgctagtccgttatcaacttgaaaaagtggcaccgagtcggtgctttt

SEQ ID NO: 6 - DNA, 79 bp, synthetic construct
  source 1..79: mol_type = other DNA
  modified_base 1: mod_base = OTHER; note = 5' amino modifier C6
  modified_base 79: mod_base = OTHER; note = dideoxyguanosine
aaaagcaccgactcggtgccactttttcaagttgataacggactagcgttaggttgacttgctatttctagctccaacc

SEQ ID NO: 7 - DNA, 80 bp, synthetic construct
  source 1..80: mol_type = other DNA
  modified_base 1: mod_base = OTHER; note = adenylpyrophosphoryl
  modified_base 80: mod_base = OTHER; note = 5' amino modifier C6
ctgctggagctagaaatagcaagtcagcataacgctagtccgttatcaacttgaaaaagtggcaccgagtcggtgctttt

SEQ ID NO: 8 - DNA, 79 bp, synthetic construct
  source 1..79: mol_type = other DNA
  modified_base 1: mod_base = OTHER; note = 5' amino modifier C6
  modified_base 79: mod_base = OTHER; note = dideoxyguanosine
aaaagcaccgactcggtgccactttttcaagttgataacggactagcgttatgctgacttgctatttctagctccagca

SEQ ID NO: 9 - DNA, 24 bp, synthetic construct
  source 1..24: mol_type = other DNA
  modified_base 1: mod_base = OTHER; note = 5' amino modifier C6
ttctaatacgactcactataggnn

SEQ ID NO: 10 - DNA, 21 bp, synthetic construct
  source 1..21: mol_type = other DNA
  modified_base 1: mod_base = OTHER; note = dideoxycytosine
  modified_base 21: mod_base = OTHER; note = 5' amino modifier C6
ctatagtgagtcgtattagaa

SEQ ID NO: 11 - DNA, 22 bp, synthetic construct
  source 1..22: mol_type = other DNA
  modified_base 1: mod_base = OTHER; note = 5' amino modifier C6
ttctaatacgactcactatagg

SEQ ID NO: 12 - DNA, 22 bp, synthetic construct
  source 1..22: mol_type = other DNA
  modified_base 1: mod_base = OTHER; note = dideoxynucleotide
  modified_base 1: mod_base = OTHER; note = dideoxynucleotide
  modified_base 22: mod_base = OTHER; note = 5' amino modifier C6
nctatagtgagtcgtattagaa

SEQ ID NO: 13 - DNA, 22 bp, synthetic construct
  source 1..22: mol_type = other DNA
ttctaatacgactcactatagg

SEQ ID NO: 14 - DNA, 20 bp, synthetic construct
  source 1..20: mol_type = other DNA
aaaagcaccgactcggtgcc

SEQ ID NO: 15 - DNA, 1839 bp, synthetic construct
  source 1..1839: mol_type = other DNA
tacctggttgatcctgccagtagcatatgcttgtctcaaagattaagccatgcatgtctnagtacgcacggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttccaagaatgttttcattaatcaagaacgaaagtcggaggttcgaagacgatcagatacctctagttccgaccataaacgatgccgaccggcgatgcggcggcgttattcccatgacccccgggcagcttccggaaaccaaagtcttgggttccggggggagtatggttcaaagctgaaacttaaaggaattgacggaagggcaccaccaggagggagccccttaatttgactcaacacgggaaacccacccggcccggacacggacaggattgacagattgatagctctttctcgattcctttggtgcatggccgttcttagttggtggagcgatttttggttaattccgataacgaacgagactctggcatgctaactagttacgcgacccccgagcggtcggcgtcccccaacttcttagagggacaagtggcgttcagccacccgagattgagcaataacaggtcttgatcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaaggcgggtcataagcttgcgttgattaagtccctccctttgtacacacccccgtcctactaccgattgatggtttagtgaggccctcggatcggccccgccggggtcggcccacggccctggcggagcgctgagaagaggtcgaacttgactattagaggaagtaaaagtcgtaacaaggtttccgtaggtgaacctgcggaaggatcatta